(19) United States
(12) Patent Application Publication
(10) Pub. No.: US 2007/0270501 A1
(43) Pub. Date: Nov. 22, 2007

(54) Medicaments for Treating Barrett's Eesophagus

(75) Inventors: Rebecca Fitzgerald, Cambridge (GB); Chih-Long Chang, Columbia, MD (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Medical Research Council, London (GB)

(21) Appl. No.: 10/588,567

(22) PCT Filed: Feb. 18, 2005
(86) PCT No.: PCT/GB05/00594

§ 371 Date: May 21, 2007

Related U.S. Application Data
(60) Provisional application No. 60/545,834, filed on Feb. 19, 2004.

(30) Foreign Application Priority Data
Feb. 19, 2004 (GB) 0403733.9

(51) Int. Cl. A61K 31/045; A61K 31/07; A61P 17/02

(52) U.S. Cl. 514/725; 514/739

(57) ABSTRACT

The present invention relates to the use of a retinoic acid antagonist in the manufacture of a medicament for the treatment or prevention of Barrett's oesophagus (Barrett's mucosa). Furthermore, the invention relates to use of a retinoic acid antagonist in the manufacture of a medicament for the treatment or prevention of ectopic development of columnar epithelium, use of such an antagonist in the conversion of columnar epithelium to squamous epithelium, in the induction or maintenance of squamous epithelium, and in the reduction of proliferation of ectopic columnar epithelium. Preferably the retinoic acid antagonist is an aldehyde dehydrogenase inhibitor such as a competitive inhibitor. Preferably the antagonist is citral.

### FIELD OF THE INVENTION

The invention relates to the application of retinoic acid antagonists to treatment of Barrett's oesophagus, and to the preparation of medicaments for such treatment.

### BACKGROUND OF THE INVENTION

It is estimated that 1 in 10 adults experiences weekly heartburn and approximately 3-12% of those people will develop the premalignant condition Barrett's oesophagus. Barrett's oesophagus is important because it is associated with a 30-fold increased risk for the development of adenocarcinoma of the oesophagus. These cancers have increased eight-fold over the past three decades, a rate exceeding that of any other solid tumour. The incidence of oesophageal adenocarcinoma is increasing particularly rapidly in the western world and is associated with a dismal prognosis.

Currently there are no effective treatment strategies for Barrett's oesophagus. Patients with Barrett's oesophagus are generally put into a surveillance programme that entails regular endoscopic examinations during which random biopsy samples are taken for assessment of dysplasia.

Barrett's oesophagus affects mainly white men, among whom the incidence of oesophageal adenocarcinoma has more than quadrupled over the past few decades. One challenge is to prevent Barrett's oesophagus from turning into oesophageal cancer. Several management strategies have been proposed to reduce mortality from cancer in Barrett's oesophagus. These include (a) normalisation (rather than mere reduction) of oesophageal acid exposure with antisecretory drugs, often in doses and combinations beyond those required to heal the symptoms and signs of reflux disease; (b) antireflux surgery, in which the weakened valve at the lower end of the oesophagus is stregthened; (c) endoscopic ablation of the metaplastic epithelium using laser or heat energy; (d) non-steroidal anti-inflammatory drugs that inhibit cyclo-oxygenase and its effects on cellular proliferation; and (e) regular endoscopic surveillance.

Patients are also encouraged make lifestyle changes to help reduce reflux which can affect/accelerate disease progression. These measures may include: eating smaller meals at regular intervals; allowing more time for food to be digested before retiring; avoiding certain foods; avoiding tight clothes and/or stooping/bending immediately after meals; losing weight and/or stopping smoking (if appropriate).

Current guidelines recommend that no therapeutic intervention is undertaken until the patient develops high grade dysplasia or early cancer. At this stage the gold standard treatment is an oesophagectomy which has an associated morbidity and mortality of approximately 5%. Furthermore, oesophagectomy itself is a complex surgical procedure requiring highly trained surgeons and the use of general anaesthesia which is itself a hazardous procedure. The costs and risks associated with this treatment are significant.

Furthermore, even the policy of non-intervention until high grade dysplasia is observed is itself problematic. One problem is the burden on healthcare providers of monitoring such patients. Another problem is the increased risk to each patient that is implicit in allowing a relatively harmless disorder to progress towards a serious and potentially fatal disorder before intervention is undertaken.

Alternative approaches to oesophagectomy include laser based ablation techniques. However, these are currently limited to research tools which are only recommended in the context of dysplasia because of the associated risks of perforation, haemmorrhage and stricturing. Furthermore, following these laser ablation therapies it is rare for the Barrett's segment to be completely reversed and hence close endoscopic follow-up is still required, which is time consuming, uncomfortable, expensive, and invasive.

Heartburn is a disorder linked to the oesophagal area, and causes discomfort and sometimes severe pain in affected individuals. This is often triggered by passage of intestinal fluids such as stomach acid into the oesophagus, and has been linked to tissue abnormalities in this region. Treatments include antacid medication, such as over-the-counter products which can be found in liquid or tablet form. These neutralize stomach acid and can be taken as needed to relieve most heartburn symptoms quickly. Because antacids are short acting and do not prevent heartburn, they are less useful for frequent or severe heartburn. Medications are available which decrease occurrence of reflux; these medications are designed to tighten the esophagus/stomach barrier or improve stomach emptying to decrease reflux. These medications are usually less effective than potent acid blockers. These medications treat acid reflux by decreasing stomach acid output. They do not work as quickly as antacids but are far more effective because they prevent acid reflux for many hours at a time. Some of these treatments are available over the counter, but the most potent and longest acting medications are available only by prescription. Severe cases may require surgery (fundoplication) to tighten the LES muscle. However, surgery is not always permanently successful, and it can cause complications. Recently, less invasive endoscopy techniques have been developed to tighten the esophagus/stomach barrier. However, the safety and effectiveness of these treatments remain enigmatic.

The present invention seeks to overcome problem(s) associated with the prior art.

### SUMMARY OF THE INVENTION

The present invention is based on the surprising finding that premalignant Barrett's oesophagus can be reversed using a retinoic acid antagonist.

We demonstrate herein the treatment of Barrett's oesophagus tissue with a retinoic acid antagonist (such as citral (Sigma)), and demonstrate that this leads to a replacement of the columnar phenotype with a squamous epithelium which has the expected cytokeratin characteristics and advantageously also has a reduction in proliferation indices. Furthermore, this effect is advantageously demonstrated in adult tissue which is clearly the most therapeutically important tissue.

Thus, the invention relates to the application of retinoic acid antagonists to treatment of Barrett's oesophagus, and to the preparation of medicaments for such treatment.

Therefore, in one aspect the invention provides use of a retinoic acid antagonist in the manufacture of a medicament for the treatment or prevention of Barrett's oesophagus (Barrett's mucosa). Formulations and preparations are described in more detail below.

Development of columnar epithelium underlies Barrett's oesophagus. The present invention advantageously permits suppression/conversion of columnar epithelium. Thus, in another aspect, the invention relates to use of a retinioic acid antagonist in the manufacture of a medicament for the treatment or prevention of ectopic development of columnar epithelium. This is preferably accomplished by conversion of the columnar epithelium to squamous epithelium. Thus, in a preferred embodiment, the invention relates to the use of a retinoic acid antagonist in the conversion of columnar epithelium to squamous epithelium.

In another aspect, the invention relates to use of a retinoic acid antagonist in the induction or maintenance of squamous epithelium.

Advantageously the invention may be used in the reduction of proliferation of columnar epithelium. Thus the invention relates to use of a retinoic acid antagonist in the reduction of proliferation of ectopic columnar epithelium. The proliferation of the columnar epithelium may be directly reduced, or the columnar epithelium may be converted to another cell type such as squamous epithelium which proliferates less than the target columnar epithelium. It is not a requirement that the cells remain columnar. The cells which are treated/targeted are columnar, whether their proliferation is reduced without conversion or via conversion eg. to squamous epithelium is not relevant so long as their proliferation is reduced. Preferably the proliferation is reduced by a process involving conversion to squamous epithelium.

In another aspect, the invention relates to a use as described above wherein the retinoic acid antagonist is an aldehyde dehydrogenase inhibitor. Preferably the aldehyde dehydrogenase inhibitor is a competitive inhibitor of aldehyde dehydrogenase.

In a preferred embodiment, the invention relates to a use as described above wherein the retinoic acid antagonist is citral.

The retinoic acid antagonist may be an antagonist of the retinoic acid receptor. Thus, in another aspect, the invention relates to a use as described above wherein the retinoic acid antagonist is a retinoic acid receptor antagonist.

In another aspect, the invention relates to a method of treating Barrett's oesophagus in a subject comprising administering to said subject an effective amount of retinoic acid antagonist. Application of the antagonist is discussed below, and is preferably applied topically, preferably as a spray. Preferably said antagonist comprises citral.

It is useful to create an in vitro/ex-vivo system for testing, assaying and assessing candidate agents according to the present invention. Thus, in another aspect, the invention relates to use of retinoic acid in the induction or maintenance of columnar epithelium. Preferably this is conducted on oesophagal tissue sample(s) in vitro. The most useful model is one which most closely mimics the clinical disease ie. the changes observed in Barrett's oesophagus. Thus, in preferred aspect, the invention relates to use of retinoic acid in the conversion of squamous epithelium to columnar epithelium.

### DETAILED DESCRIPTION OF THE INVENTION

Heartburn/Acid Reflux

Heartburn is a burning pain behind the lower breastbone that may radiate upward toward the neck. It may also include the sensation of food or liquid coming up into the throat or mouth (regurgitation), especially when bending over or lying down. Pain can also be felt at the same level in the mid-line of the back. These symptoms may be accompanied by a bitter or acid taste.

A sphincter known as the lower esophageal sphincter (LES), is located at the end of the esophagus and opens during swallowing to allow food to pass into the stomach. The LES muscle then closes quickly to prevent the return (reflux) of food and stomach juices back into the esophagus. However, the LES muscle does not always work perfectly. Gastroesophageal reflux occurs when the LES muscle either relaxes inappropriately or is weak. This allows stomach juices to back up, or reflux, into the esophagus, creating heartburn. Recurrent heartburn is the most common symptom of a condition called gastroesophageal reflux or acid reflux and a condition known as Gastroesophageal reflux disease, which occurs when the lower oesophageal sphincter allows the stomach content to leak back frequently into the oesophagus.

There are several factors that influence the frequency and severity of acid reflux: the ability of the LES muscle to open and close properly, the type and amount of stomach juices that reflux up into the esophagus, the ability of the stomach to empty properly, the clearing action of the oesophagus, the acid-neutralizing effect of saliva and other factors.

Acid reflux can sometimes result in serious complications. Oesophagitis, an inflammation of the oesophagus that can lead to oesophageal bleeding or ulcers, can occur as a result of frequent exposure of the oesophagus to stomach acid. In addition, a narrowing or partial closure (stricture) of the lower oesophagus may occur, interfering with a person's ability to swallow. Some people develop a condition known as Barrett's oesophagus, a change in the cells of the tissue lining the bottom of the oesophagus that can increase the chance of developing cancer. In most cases, individuals with Barrett's oesophagus have to be monitored with periodic upper gastrointestinal endoscopy and biopsies. Lung problems can also develop when reflux causes stomach fluid to overflow into the breathing tubes. This often occurs at night when a person is lying down and may cause wheezing, bronchitis and pneumonia.

Other possible problems caused by acid reflux include inflammation of the throat, voice box and airways.

The present invention finds application in countering the consequential effects of heartburn/acid reflux, which include histological and related changes in oesophagal tissue, in particular Barrett's mucosa/Barrett's oesophagus.

Barrett's Oesophagus

The incidence of oesophageal adenocarcinoma is increasing rapidly. It is preceded by the metaplastic condition Barrett's oesophagus in which squamous tissue becomes columnar by an unknown mechanism.

First identified in the 1950's by the surgeon Norman Barrett, Barrett's oesophagus is when the stratified squamous epithelium that normally lines the distal oesophagus is replaced by an abnormal columnar epithelium that has intestinal features. The abnormal epithelium (ie. the specialised intestinal metaplasia) usually shows evidence of DNA damage that predisposes to malignancy, and most oesophageal adenocarcinomas seem to arise from this metaplastic tissue.

It is thought that Barrett's oesophagus develops as a consequence of chronic gastro-oesophageal reflux disease (GORD) in which stomach juices reflux into the Oesophagus. Unlike the stomach, the oesophagus does not have a protective lining, so when it is repeatedly exposed to stomach acids it may become inflamed and painful (oesophagitis). On occasion contents from the duodenum, particularly bile, may also reflux into the oesophagus, via the stomach. A mixture of stomach and duodenal contents in the oesophagus is even more damaging than acid alone. The oesophagus usually heals with time and the lining returns to normal, but occasionally, and particularly if bile is present, the lining may heal abonormally, causing it to appear more like the lining of the stomach or small intestine. This type of lining is unstable when present in the oesophagus and complications may develop. Precisely how or why the change occurs is not known at the molecular level, but is discussed in more detail below.

Barrett's oesophagus is usually discovered during endoscopy performed to evaluate the symptoms of reflux disease and is recognisable because the dull red of the metaplastic columnar epithelium contrasts sharply with the pale glossy normal squamous lining.

The condition is often symptomless. Most people diagnosed with Barrett's Oesophagus will have been examined due to symptoms associated with gastro-oesophageal reflux, which causes a burning pain in the gullet, usually following a meal or when bending or lying down. Other common symptoms include a salty taste at the back of the mouth (termed water brash), hoarseness due to acid damaging the vocal cords and chest pain.

In a minority of sufferers, Barrett's Oesophagus can lead to complications such as ulcers in the gullet, bleeding, difficulty in swallowing due to a narrowing of the gullet (stricture), and cancer (oesophagal adenocarcinoma).

Barrett's oesophagus is classified as long segment or short segment, depending on whether or not the specialised intestinal metaplasia extends 3 cm or more above the gastro-oesophageal junction. Among patients who have endoscopy for symptoms of reflux, long segment Barrett's oesophagus is found in 3-5% and short segment disease in 10-15%. Although it is not clear whether long and short segment Barrett's oesophagus have the same pathogenesis and risk for malignancy, the two conditions are managed similarly. The present invention applies equally to both forms, with any necessary adjustments eg. to dosage/application being within the abilities of the person skilled in the art.

Developmental Pathways in Barrett's Oesophagus

The columnar lined oesophageal segment probably develops to its full length over a period of weeks. This metaplastic change is rarely observed in vivo and hence natural history studies to determine the Barrett's cell of origin have not been possible. Three possibilities for the tissue of origin for Barrett's metaplasia have been hypothesized. Firstly, the de novo metaplasia theory proposes that pluripotential stem cells of the exposed papillae in inflamed squamous mucosa are damaged leading to abnormal differentiation along a Barrett's cell lineage. However, this would seem unlikely since recent data has demonstrated that the inter-basal layer (between the papillae) of the oesophagus is enriched for stem cells which give rise to daughter cells through populational asymmetry. The second theory is the transitional zone metaplasia theory, which suggests that pluripotential stem cells at the gastro-oesophageal junction (transitional zone) colonise the distal oesophagus in response to noxious luminal agents. However, this is not supported by animal experiments in which there is a mucosal defect separating the distal oesophagus from the transitional zone. These animals can still develop columnar oesophagus. Thirdly, the duct-cell metaplasia theory suggests that stem cells located in the glandular neck region of oesophageal submucosal gland ducts selectively colonise the oesophagus in response to squamous mucosal damage. The basis for this mechanism is the ulcer-associated cell lineage.

The mechanism by which a cell commits and proceeds to its full differentiation status is not fully understood. However, it is increasingly recognised that epithelial-stromal interactions are important and interestingly, in order to achieve asymmetrical stem cell divisions of the oesophageal epithelium, it is necessary to reconstitute the oesophageal squamous keratinocytes on denuded connective tissue derived from the oesophagus.

During embryogenesis the oesophagus undergoes a columnar to squamous cell transition at 18 weeks gestation. Hence it is possible that alterations in the genes responsible for this physiological transition may be abnormally reactivated leading to the development of metaplastic Barrett's oesophagus in adulthood.

Here we surprisingly demonstrate that mature, adult human squamous tissue treated with retinoic acid ex vivo can induce columnar differentiation mimicking Barrett's metaplasia. The metaplastic tissue arises from cells contined within the mesenchymal compartment, has some characteristics of the ulcer associated cell lineage and does not require epithelial cell signalling or cell proliferation. These results suggest a novel mechanism for the development of Barrett's oesophageal epithelium that can be recapitulated in vitro. Thus the invention provides a unique model system in which to investigate and assay the molecular mechanisms underlying oesophageal metaplasia.

Columnar-Squamous Phenotypes

The columnar (sometimes referred to as ‘glandular’) and squamous tissue types are easily distinguished by the person skilled in the art with reference to the current histological resources. Where appropriate, the particular characteristics of oesophagal tissue should be taken into account. In particular, cell morphology should be considered. Preferably assessment of molecular markers ascribed to the different cellular types is also employed. Examples of such assessments are provided herein and include cytokeratin assessment for squamous epithelium. Furthermore, any of the molecular markers presented in the Examples section may be employed, either alone or in combination. Other molecular markers known in the art but not demonstrated herein may equally be used if desired, either alone, in combination with one another or in combination with one or more of those markers demonstrated herein.

Applications

One of the principal applications of the present invention is in the treatment or prevention of Barrett's oesophagus (Barrett's mucosa). In particular the invention is effective against ectopic development of columnar epithelium which is regarded as an initial step and a characterising feature of Barrett's Oesophagus.

The present invention also finds preventive application in related conditions such as heartburn, ulceration of the gullet, acid reflux and similar complaints. It is important to note that these conditions may be considered as occurring prior to or ‘upstream’ of Barrett's oesophagus, and prevention of these ‘upstream’ disorders themselves is not asserted for the present invention, it is prevention/treatment of the histologically defined stage(s) such as Barrett's s oesophagus itself which are the object of application of the present invention to patients presenting with ‘upstream’ disorders such as acid reflux/heartburn. These ‘upstream’ conditions may be indicia of a need for preventative treatment, or indeed an indication of risk of development of Barrett's oesophagus. However, it is the prevention/treatment of Barrett's oesophagus which is a focus of the present invention rather than, for example, prevention of acid reflux itself.

Furthermore, the invention finds application in more clinically advanced conditions such as dysplasia in Barrett's oesophagus, oesophagal adenocarcinoma and related conditions. The invention may be of direct application in said disorders, and/or may be used in the treatment of the underlying Barrett's oesophagus which may be masked by dysplasia and/or adenocarcinoma. Preferably the invention is used in the treatment of the underlying Barrett's oesophagus.

Preferably the treatments/compositions of the present invention are useful independent of cellular proliferation of the target cells, preferably their action is by transdifferentiation of said target cells.

The model system of the present invention finds application in the testing, assay, validation and assessment of retinoic acid antagonists for their ability to drive the therapeutically important columnar-squamous transition.

Retinoic Acid and Related Agents

Agents of the present invention are those molecular entities connected with retinoic acid signalling.

In the prior art, the availablility and limited use of retinoic acid antagonists has been driven by the interest in retinoic acid agonists for the treatment of leukaemias, psoriasis, acne or diabetes. The antagonist compounds have previously been used to test the specifity of agonist effects in vitro and as possible antidotes to retinoic induced toxicity. The present invention surprisingly teaches clinical applications of retinoic acid antagonists.

In therapeutic aspects of the present invention, such as for inducing squamous phenotype from columnar material, agents are preferably retinoic acid antagonists which may be any entity capable of suppressing, inhibiting, limiting, downregulating, capping, reducing, preventing, ameliorating, ablating, or otherwise alleviating or countering the effect(s) of retinoic acid. These agent(s) may act on retinoic acid or may act on another molecule or a number of molecule(s) to produce or exert their effect(s), for example they may produce a blockage in the retinoic acid signalling pathway. For example, a retinoic acid antagonist may act upstream to prevent retinoic acid being made in vivo. A retinoic acid antagonist may be an inhibitor of the aldehyde dehydrogenase enzyme, preferably a competitive inhibitor of said enzyme. A retinoic acid antagonist may be a retinoic acid receptor antagonist.

Two classes of retinoid receptors are currently known. The retinoic acid receptors (RAR α,β,γ) have all-trans retinoic acid (ATRA) as their natural ligand, and the retinoid-X-receptors (RXR α,β,γ) for which 9-cis retinoic acid has been proposed as the endogenous ligand. Preferably the agents of the present invention are antagonists of RAR or RAR mediated pathways.

A diverse group of compounds has emerged which are antagonists of retinoic action medicated by the RAR family of receptors. Exemplary compounds are discussed in more detail below.

The general structure of RAR antagonist compounds is shown in Johnson et al Bioorganic and Medicinal Chemistry 7, 1999:1321-1338. This is incorporated herein by reference. In particular the structures of the RAR antagonist compounds are incorporated herein with particular regard to table 1 of this publication. Compounds 3-6 are low affinity antagonists which require their use at concentrations in excess of 100 to 1000 fold of the natural hormone ATTRA to inhibit retinoid induced biological activity. In contrast, compounds 7 and 8 have been shown to be potent RAR antagonists with binding affinities comparable to the natural ligand ATRA. Compounds 7 and 8 are therefore preferred agents of the present invention.

Using the known crystal structure for RA a modelling approach was used to identify novel compounds which may act as RAR antagonists. These were identified from a screening database of 150,000 compounds. From this, approach 2 novel antagonists were discovered. Thus, these two RAR antagonists disclosed in Schapira et al, PNAS 2000, 97:1008-1013 are specifically incorporated herein by reference. These two RAR antagonists are preferred agents of the present invention.

Citral

Citral (3,7-dimethyl-2,6-octadienal) is a highly preferred agent of the present invention.

Citral is known as a flavouring and fragrance agent. A series of microsomal cytochrome P450 (P450) isozymes is known to be involved in all-trans-retinoid metabolism, including the conversion of all-trans-retinal to all-trans-retinoic acid. Evidence has been obtained that citral is an effective mechanism-based inactivator of isozyme 2B4, with a KI of 44 microM as determined by the oxidation of 1-phenylethanol to acetophenone, and by isozyme 1A2 in the oxidation of all-trans-retinal to the corresponding acid and by isozyme 2B4 in the 4-hydroxylation of all-trans-retinol and retinoic acid, (Raner et al 1996, Mol Pharmacol 49:515-22).

Reports of the in vivo metabolism of citral suggest that a primary route of metabolism is conversion to the corresponding acid species, probably by aldehyde dehydrogenases (ALDH), (Boyer C S and Petersen D R Drug Metab Dispos 1991;19:81-6).

Dihydronaphthalene Based RAR Antagonists

A large number of agonists and antagonists for Retinoic acid (RA) have been produced based on dihydronaphthalene. The effectiveness of antagonists depends on the introduction of heteroatoms in the bicyclic ring system of the potent dihydronaphthalene RAR antagonist, and the variation of the pendant aromatic group.

Binding, transcriptional and in vivo assays revealed that the 2,2-dimethylthiochromene analogue 59, and the 2,2-dimethylthiochromene derivative 85, were the most effective in blocking retinoid agonist induced acitivity (Johnson A T et al Bioorg Med Chem 1999, 7:1321-38—incorporated herein by reference, with particular reference to the further structural detail presented for the above described compounds). Derivatives 59 and 85 are preferred agents of the present invention.

Further preferred agents include:
1. AGN194310 A high affinity pan-retinoic acid receptor (RAR) antagonist, K_{d} for binding to RARs=2.5 nM;
2. AGN194431 Antagonist with predominant activity at RARβ and RARγ with a K_{d} value of 70 nM at RARγ;
3. AGN194301 an RAR antagonist; and
4. AGN 193109 RAR antagonist.

Each of these agents 1-4 is available from Allergan Inc.
5. RARα selective antagonist Ro 41-5253 (available from Novartis, Basel)
6. ST2065—a retinoid related molecule with antagonistic properties. (available from Sigma-Tat Industrie Farmaceutiche Riunite s.p.a., Italy)
   Diazepinylbenzoic Acid Derivatives

Several diazepinylbenzoic acid derivatives are known as retinoid X receptor (RXR) antagonists on the basis of inhibitory activity on retinoic-induced cell differentiation of human promyelocytic leukaemia cells. 4-(5H-2,3-Dimethyl-2,5-hexano)-5-n-propyldibenzo[b,e][1,4]diazepin-11-yl)benzoic acid (HX603, 6c) is an N-n-propyl derivative of an RXR pan-agonist HX600 (6a) and exhibits selective antagonistic activity. Other chemical derivatives have also been sythesised with similar antagonistic activity—see Ebisawa et al Chem Pharm Bull, 1999, 47: 1778-86 (incorporated herein by reference with particular regard to the chemical information on the RXR antagonists described)—and may therefore find application as agents of the present invention. LG100754 is a further RXR antagonist which may find application in the present invention (see Ebisawa et al Chem Pharm Bull, 1999, 47: 1778-86 as above).

Benzoic Acid Based Compounds

Eisai Co., Ltd have developed RAR antagonists based on heterocyclic ring-containing benzoic acids. Structure activity relationships indicate that both an N-substituted pyrrole or pyrazole (1-position) and a hydrophobic region with these linked by a ring system, were indispensable for effective anatagonism. All 3 of the most effective compounds,
4-[4,5,7,8,9,10-hexahydro-7,7,10,-10-tetramethyl-1-(3-pyridylmethyl)anthra[1,2-b]pyrrol-3-yl]benzoic acid (31);
4-[4,5,7,8,9,10-hexahydro-7,7,10,10-tetramethyl-1-(3-pyridylmethyl)-5-thiaanthral[1,2-b]pyrrol-3-yl]benzoic acid (40), and
4-[4,5,7,8,9,10-hexahydro-7,7,10,10-tetramethyl-1-(3-pyridylmethyl)anthra[2,1-d]pyrazol-3-yl]benzoic acid (55),
   all of which possess a 3-pyridylmethyl group at the five-membered ring nitrogen atom.

These three compounds are preferred agents of the present invention and are described in detail in Yoshimura H et al, J Med Chem 1995, 38:3163-73 which is incorporated herein by reference specifically for the chemical information described above. The compounds are available from Tsukuba Research Laboratories, Eisai Co., LTD., Ibaraki, Japan.

A most highly preferred retinoic acid antagonist according to the present invention is citral (available from Sigma).

Preferably the agent according to the invention is not SELBEX® (terprenone or geranylgeranylacetone) which is preferably expressly disclaimed. Preferably the agent is not any agent of the chemical structure as disclosed in WO02/098398, which are preferably expressly disclaimed with reference to said document. More preferably the agent of the invention is not a compound described in U.S. Pat. No. 4,169,157.

Agents—Model System Aspects

In model system aspects of the invention, such as for inducing columnar phenotype from squamous material, agents are preferably retinoic acid agonists which may be any entity capable of increasing, enhancing, initiating, augmenting, supplementing, stimulating, maintaining, amplifying, expanding or otherwise facilitating or promoting the effect(s) of retinoic acid. These agent(s) may act on retinoic acid or may act on another molecule or a number of molecule(s) to produce or exert their effect(s). A retinoic acid agonist may be a retinoic acid receptor agonist.

A range of selective RARα agonists is available from Novartis (Basel).

There is a range of ST compounds (available from Sigma-Tau Industrie Farmaceutiche Riunite s.p.a., Italy) which are largely retinoic acid receptor agonists. These chemical are related to the CD347 prototype which is a promising class of cytotoxic compounds known as adamantly-retinoids (Cincinelli et al J Med Chem 2003; 46: 909-912—incorporated herein by reference in particular for further details of the chemical structure of these agonists including CD347). CD347 has a chemical structure similar to retinoic acid and was originally developed as a selective agonist of the retinoic acid receptor RARγ. These drugs have been developed as anti-leukemic agents. Though the mechanism of action is still largely obscure it appears to act differently to many chemotherapeutics and apoptotic agents. Recent evidence suggests that the mitochondrion may be an important target, causing opening of the mitochondrial transition pore, release of cytochrome C into cytosol and subsequent activation of caspase proteolytic cascade. De novo protein synthesis does not seem to be required and is associated with MAPK activation. In spite of in vitro and in vivo activity, CD437 has limited clinical potential given the narrow therapeutic window and a relatively unfavourable pharmacokinetic profile. However, it finds application in the model system aspects of the invention where such properties are less problematic. The RAR agonist ST2065 has a heterocyclic ring in the centre of the molecule. ST1926 is more powerful than CD437 and is a bona fide retinoid related molecule which is currently undergoing clinical development to be administered as an oral compound (Garattini et al, Blood September 2003—Incorporated herein by reference in particular for the further chemical structure of CD437).

R115866 inhibits all-trans Reinoic acid metabolism and hence increases RA levels. This approach was developed because high-metabolism can impair the biological efficacy of RA. R115866 is a novel inhibitor of the cytochrome P450 (CYP)-mediated metabolism of RA and is described in detail in Stoppe et al J of Pharm and Exp Therapeutics 2000 which is incorporated herein by reference with particular regard to the information regarding R115866.

A preferred retinoic acid agonist according to the invention is retinoic acid such as all-trans retinoic acid.

Pharmaceutical Compositions

The present invention also provides a pharmaceutical composition comprising a therapeutically effective amount of the agent(s) and/or modulator(s) of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavouring agents may be provided in the pharmaceutical composition. Examples of preservatives include sodium benzoate, sorbic acid and esters of p-hydroxybenizoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a buccal/oesophagal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Where the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

If the agent/modulator is a protein, then said protein may be prepared in situ in the subject being treated. In this respect, nucleotide sequences encoding said protein may be delivered by use of non-viral techniques (e.g. by use of liposomes) and/or viral techniques (e.g. by use of retroviral vectors) such that the said protein is expressed from said nucleotide sequence.

In a preferred embodiment, the pharmaceutical of the present invention is administered topically. Hence, preferably the pharmaceutical is in a form that is suitable for topical delivery.

Administration

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The retinoic acid antagonists (or agonists for the model system aspects of the invention) can be administered orally and/or topically. Teachings on administration may be found in Standeven AM Fundam Appl Toxicol 1996, 91.

A topical route of administration, such as a spray, is preferred. One advantage of this administration is to minimise systemic side-effects

The components of the present invention may be administered alone but will generally be administered as a pharmaceutical composition—e.g. when the components are is in admixture with a suitable pharmaceutical excipient, diluent or carrier selected with regard to the intended route of administration and standard pharmaceutical practice.

For example, the components can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyriolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray, buccal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, intraoesophagal, subcutaneous, ophthalmic (including intravitreal or intracameral), transdermal, rectal, buccal, vaginal, epidural, sublingual. Preferably the administration is intraoesophagal.

In a preferred aspect, the pharmaceutical composition is delivered topically. In a highly preferred aspect, the pharmaceutical composition is delivered as a spray. In a most highly preferred aspect, the pharmaceutical composition is delivered topically as a spray.

It is to be understood that not all of the components of the pharmaceutical need be administered by the same route. Likewise, if the composition comprises more than one active component, then those components may be administered by different routes.

If a component of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) of the present invention can be administered in the form of a suppository or pessary, or it may be applied topically in the form of a gel, hydrogel, lotion, solution, cream, ointment or dusting powder. The component(s) of the present invention may also be dermally or transdermally administered, for example, by the use of a skin patch. They may also be administered by the pulmonary or rectal routes. They may also be administered by the ocular route.

For application topically to the skin, the component(s) of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Pharmaceutical Combinations

The agent of the present invention may be administered with one or more other pharmaceutically active substances. By way of example, the present invention covers the simultaneous, or sequential treatments with an agent according to the present invention and one or more steroids, analgesics, antivirals or other pharmaceutically active substance(s).

It will be understood that these regimes include the administration of the substances sequentially, simultaneously or together.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending upon the need, the agent may be administered at a dose of from 0.01 to 30 mg/kg body weight, such as from 0.1 to 10 mg/kg, more preferably from 0.1 to 1 mg/kg body weight.

A preferred formulation of Citral aims for 20 mM application. Toxicology studies on Citral were conducted by the National Cancer Institute because of its widespread use in foods, beverages, cosmetics, and other consumer products and its structure as a representative beta-substituted vinyl aldehyde. When citral was fed to rats in toxicology studies it was administered orally over a certain dose range (NTP toxicology and carcinogenesis studies of citral (microencapsulated) (CAS No. 5392-40-5) in F344/N rats and B6C3F1 mice (feed studies), Natl Toxicol Program Tech Rep Ser 2003,505:1-268). Clearly attention must be paid to this when designing the actual dosing scheme as necessary.

Formulation

The component(s) of the present invention may be formulated into a pharmaceutical composition, such as by mixing with one or more of a suitable carrier, diluent or excipient, by using techniques that are known in the art.

Pharmaceutically Active Salt

The agent of the present invention may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

Treatment

It is to be appreciated that all references herein to treatment include one or more of curative, palliative and prophylactic treatment. Preferably, the term treatment includes at least curative treatment and/or prophylactic treatment.

The treatment may be of one or more of those disorders mentioned herein, or related complaint.

The time course of treatment is preferably determined by a physician. Exemplary time course is 48-72 hours, preferably 72 hours. When the retinoic acid antagonist is citral, preferably the treatment is for approximately 48-72 hours, preferably 72 hours.

Therapy

The agents/modulators identified by the methods of the present invention may be used as therapeutic agents—i.e. in therapy applications.

As with the term “treatment”, the term “therapy” includes curative effects, alleviation effects, and prophylactic effects.

The therapy may be on humans or animals.

The therapy can include the treatment of one or more of those disorders mentioned herein, or related complaint.

The invention finds application in therapeutic industry such as application to, and/or preparation of medicaments for, patients with Barrett's oesophagus. If the Barrett's mucosa is completely removed then this advantageously eliminates the need for endoscopic screening. Furthermore, such a treatment/medicament may also be applied as a chemopreventive agent to that population of patients with heartburn.

Without wishing to be bound by theory, it is possible that replacement of the columnar phenotype with a squamous epithelium according to the present invention is occurring through a process of transdifferentiation or via a cell repopulation process and naturally it may be beneficial to understand the signalling pathways involved in more detail. The mechanism of action of retinoic acid antagonists such as citral is not fully understood but it is thought that it may compete with the aldehyde dehydrogenase enzyme required to convert retinoic acid into its active metabolites.

Advantageously the effects of other compounds implicated in these processes, such as retinoic acid receptor antagonists may be conveniently studied using the ex vivo system of the present invention.

In addition, the present invention relates to in vitro development of oesophageal metaplasia via application of retinoic acid.

In another aspect, the invention relates to the modulation of mesenchymal-epithelial transition using the techniques and/or conditions disclosed herein.

In another aspect, the invention relates to induction of a columnar lined mucosa from mature adult squamous oesophageal tissue in an ex vivo model system such as a human ex vivo system. It is disclosed herein that exposure to all-trans retinoic acid mimics morphological and molecular features of Barrett's oesophagus, apparently via a mesenichymal to epithelial transition resembling the ulcer-associated cell lineage.

Previous work on the cell signalling pathways involved in retinoic acid induced columnar differentiation suggest that the first step in the mesenchymal to epithelial transition involves cell migration, followed by differentiation and cell cycle arrest; a process called cell condensation. Advantageously the system of the present invention embraces condensation.

The retinoid receptors bind to recognition sequences (RAREs) within the controlling region of target genes to activate or suppress their activity. Hence, a dynamic balance of multiple signals from diverse pathways may be reprogrammed to signal the end of stem cell renewal and the onset of lineage commitment. The genetic controls include extracellular matrix molecules, cell adhesion molecules and many genes associated with developmental pathways including those involving TGF-β superfamily members such as bone morphogenic proteins (BMPs), homeo-domain (cdx and hox), IGF, Notch, Hedgehog and Wnt proteins. The in vitro model of the present invention advantageously enables the specific pathways involved in oesophageal metaplasia to be elucidated. Activation of specific pathways may also be germane to understanding why only a subset of retinoic acid treated ex vivo cultures, and reflux patients, develop Barrett's oesophagus. Resolution of these issues is advantageously made possible by the model of the present invention.

CYP26

Addition of an RA antagonist such as citral to normal squamous epithelium results in a depression of CYP26A1 activity. Time course studies reveal that this is a pulse effect and so a sustained depression of CYP26A1 activity may require a sustained application of antagonist such as citral. CYP26A1 is involved in RA degradation.

In another aspect the invention relates to use of a retinoic acid antagonist in the downregulation of CYP26A1.

In another aspect the invention relates to use of a retinoic acid antagonist in the manufacture of a medicament for the downregulation of CYP26A1.

In another aspect the invention relates to use of an inhibitor of CYP26A1 activity in the manufacture of a medicament for the treatment or prevention of Barrett's oesophagus (Barrett's mucosa).

In another aspect the invention relates to a method of treating Barrett's oesophagus in a subject comprising administering to said subject an effective amount of a CYP26A1 inhibitor.

The CYP26A1 antagonist may be any entity capable of suppressing, inhibiting or downregulating CYP26 activity and/or expression, and may be in the form of RNAi or other such moiety. Preferably the antagonist used to downregulate CYP26A1 (or CYP26A1 inhibitor) is citral.

In another aspect the invention relates to a method of inhiniting retinoic acid degredation by inhibition of CYP26A1. Furthermore, in another aspect the invention relates to a method of increasing retinoic acid concentration by inhibiting CYP26A1.

In another aspect, is may be desirable to produce an enhanced CYP26A1 activity for a period, perhaps in order to reduce or depress RA levels. In this embodiment the withdrawl of antagonist such as citral may advantageously provide the desired effect, or a direct enhancement such as by overexpression or gene therapy of CYP26A1 may be preferred.

It will be apparent to the reader that due to the effects demonstrated in squamous epithelium, that this embodiment may advantageously be applied to the model system aspects of the invention.

Cdx2

Increased Cdx2 activity can lead to increased RA activity. Thus according to the present invention suppression, downregulation or inhibition of Cdx2 activity is advantageous in the reduction of RA signalling and therefore in the prevention or treatment of Barrett's oesophagus. Cdx2 activity can be suppressed by any suitable means known in the art such as RNAi suppression of expression of Cdx2, preferably in an localised topical application for use in the oesophagus.

Thus in another aspect the invention relates to the downward modulation of Cdx2 activity in the prevention or treatment of Barrett's Oesophagus.

In another aspect the invention relates to use of a Cdx2 inhibitor in the manufacture of a medicament for the prevention or treatment of Barrett's Oesophagus.

In another aspect the invention relates to use of an inhibitor of Cdx2 activity in the manufacture of a medicament for the treatment or prevention of Barrett's oesophagus (Barrett's mucosa).

In another aspect the invention relates to a method of treating Barrett's oesophagus in a subject comprising administering to said subject an effective amount of a Cdx2 inhibitor.

Further Aspects: Mechanism of Action

Without wishing to be bound by theory, with reference to the examples section, it appears that the Cdx2 can up-regulate the RA synthesizing enzyme and restore RA biosynthesis and there are two RARE sequences in the promoter of CYP26A1. In one scenario, Cdx2 will increase RA and would in turn be expected to up-regulate CYP26A1 in order to degrade RA in normal tissues to maintain homeostasis. However, in BE the CYP26A1 is downregulated and so RA biosynthesis will remain increased. Moreover, it may be that acid is the initial stimulus for upregulation of Cdx2 in vivo. In the ex vivo system of the invention, Cdx2 is bypassed as we teach adding exogenous RA which will then advantageously affect key differentiation processes directly.

Down-regulation of CYP26A1 in early Barrett's epithelia may be by any suitable method such as mutation, promoter methylation, RNAi or other technique known in the art.

The invention may be advantageously combined with other retinoid pathway enzymes responsible for the disease process such as like CRBP-1, ALDH family enzymes.

The relationship between Cdx2 expression, retinoic biosynthesis and acid stimulation on epithelium may advantageously be determined in individual systems for example by using primary cells and cell lines stably transfected with Cdx2 promoter/EGFP reporter vectors to determine whether long-term acid stimulation can induce Cdx2 expression and in turn enhance retinoic acid biosynthesis. In addition, stably over-expressing Cdx-2 in epithelial cell cultures (both monolayer and organotypic cultures) may advantageously be used to determine the effect of this on the cell phenotype.

The protein transduction polypeptide (poly-Arginine) may be used to deliver the Cdx2 protein into tissues, for example those cultured in an organ culture system. This approach can also be combined with a fluorescent marker and confocal imaging in order to determine the cell of origin for the metaplastic cell.

The present invention will now be described, by way of example only, in which reference will be made to the following figures:

### BRIEF DESCRIPTION OF THE FIGURES

FIG. 1 Ex vivo culture of squamous oesophageal biopsies in retinoic acid induces a columnar phenotype with the immunohistochemical characteristics of native columnar lined (Barrett's) oesophagus. NE is normal squamous oesophageal epithelium which has been cultured ex vivo for 48 hours in the absence or presence of retinoic acid (−RA, +RA). BE is uncultured Barrett's oesophageal epithelium for comparison. Routine haematoxylin and eosin (H&E) has been performed as well as immunohistochemistry using immunoperoxidase labelled CK8/18,13 and 7, villin and TFF1 antibodies. Magnification ×100.

FIG. 2 Ex vivo culture of Barrett's oesophageal biopsies in citral induces a squamous phenotype with the immunohistochemical characteristics of native squamous oesophagus. NE is normal squamous oesophageal epithelium which has been cultured ex vivo for 48 hours in the absence or presence of citral. BE is uncultured Barrett's oesophageal epithelium for comparison. Routine haematoxylin and eosin (H&E) has been performed as well as immunohistochemistry using immunoperoxidase labelled CK8/18,13, 7 and 14. Magnification ×100.

FIG. 3 The alteration in phenotype is not dependent on cell proliferation. There is very little BrdU incorporation in retinoic acid (RA) treated squamous oesophageal tissue (NE) which has undergone glandular differentiation, compared to the cultured native Barrett's oesophagus tissue (BE). Immunohistochemical analysis of Ki-67 confirmed the lack of proliferation in the squamous treated tissue and enhanced p21 expression suggests RA induced cell cycle arrest. Magnification ×100.

FIG. 4 Mesenchymal tissue alone is sufficient for the induction of glandular metaplastic transformation ex vivo, RT-PCR confirmed that presence of retinoic acid (RA) induced the glandular epithelial markers CK 8/18 and 7 with reduction of the squamous epithelial CK13 in a full thickness squamous epithelial biopsy (NE). The mesenchyme alone (M) cultured in the absence of RA did not express any epithelial cell markers CK13, 8/18 and 7, but following RA treatment CK 8/18 and 7 were again induced. N was a negative control using water in the RT mix, BE was a full thickness biopsy from Barrett's oesophagus, and GAPDH confirmed equivalent cDNA loading

FIG. 5 The temporal sequence of the molecular changes in the squamous epithelium treated with retinoic acid. The morphological (H&E) and immunohistochemical (peroxidase labelled CK7, p63, vimentin and CK14) characteristics of biopsies cultured for 24, 32 and 48 hours in the presence of retinoic acid are demonstrated. The characteristics of the squamous epithelium at 0 hours, prior to culture, are shown for comparison.

FIG. 6a The co-localisation of CK8/18 and vimentin expression after 24 hours which then compartmentalises by 32 hours suggests a mesenchymal origin for the glandular epithelium. CK8/18 (green) and vimentin (red) are viewed using the confocal microscope over time (24, 32 and 48 hours). Co-expression is seen by dual labelling at 24 hours, which separates into glands (CK8/18 positive) and surrounding stroma again at 32 hours.

FIG. 6b Native Barrett's oesophagus biopsies have immature deep glands which resemble the retinoic acid induced changes in the system of the present invention. Biopsies have been stained with hematoxylin and eosin and viewed with the light microscope, or with TO-PRO-3 DNA stain (blue), CK8/18 (green), vimentin (red) and dual labelling. The mature glands (arrowhead) in this representative biopsy have nuclei which are configured in a clear glandular arrangement and stain with CK8/18 with no expression of vimentin. In contrast, there are more immature areas (arrow) in which the nuclei are more loosely palisaded with weak expression of CK8/18 and speckled vimentin in the same areas.

FIGS. 7a and 7b Show photomicrographs of induced squamous epithelium from Barrett's glandular tissues and normal squamous tissues.

FIG. 8 shows a plot of RA concentrations by cell line.

FIG. 9 shows a plot of RA concentrations by tissue type.

FIG. 10 shows a western blot and a plot of RA concentrations by cell line/transfection regime.

FIG. 11 shows RT-PCR results.

### EXAMPLE 1

### Retinoic Acid Induces Glandular Oesophageal Mucosa Ex Vivo

To demonstrate that retinoic acid can induce differentiation in squamous oesophagus, endoscopic biopsies are cultured in an organ culture system.

Patients and tissues Patients were recruited prospectively from Addenbrooke's Hospital, Cambridge with approval by the Local Research Ethics Committees. Patients were being endoscoped for a variety of clinical indications and as pail of a Barrett's surveillance programme. All patients with Barrett's oesophagus, used for control experiments, had an endoscopically visible columnar lined segment and a histopathological diagnosis of specialised intestinal metaplasia. All squamous oesophageal samples (n=40, mean age 62 yrs, 1.2M:1F; n=18 controls cultured without retinoic acid mean age 69 yrs, 1M:1F) were taken from at least 2 cm above the squamo-columnar junction.

Organ Culture

Endoscopic samples were cultured in organ culture media (Medium 199 containing 2 mM glutamine) at a liquid-oxygen (95%) interface as previously described (Fitzgerald et al. J. Clin. Invest. vol 98, 2120-2128 (1996)). Media was supplemented with 100 IU All Trans retinoic acid (Sigma, Poole, UK) for a maximum of 72 hours. For the BrdU incorporation experiments 30 uM was added to the media for the duration of the experiment. In order to separate the epithelial and mesenchymal compartments the biopsy tissue was incubated in balanced salt solution with 2 U/ml dispase (Gibco) for an hour, and then washed vigorously to remove the epithelium.

Histochemical Stains and Immunohistochemistry.

Tissues were fixed in 10% formalin and processed for paraffin embedding. 5-um sections were stained with hematoxylin and eosin. For immunostaining the following primary mouse monoclonal antibodies raised against human CK7 (1:50), CK13 (1:200), CK8/18 (1:40) CK14 (1:40), vimentin (1:50), p63 (1:25) (Novocastra, Newcastle Upon Tyne, UK), villin (1:750) (Chemicon, Harrow, UK) and Ki-67 (1:100) (DakoCytomation, Ely, UK) were used. Anti-BrdU (1:40) (AbCam, Cambridge, UK) was used following an antigen retrieval step in 2N HCl. We used the avidin-biotin and peroxidase method and a hematoxylin counterstain. Negative controls were performed for each slide by omission of the primary antibody.

Immunoflurescence.

Sections were incubated with monoclonal rabbit anti-vimentin (1:40) (Santa Cruz biotechnology, Santa Cruz, Calif.) and monoclonal mouse anti-CK8/18 (1:40) (Novocastra, Newcastle Upon Tyne, UK) simultaneously. The secondary antibodies used were FITC-conjugated anti-mouse antibody (Vector, Burlingame, Calif.) and Texas red-conjugated anti-rabbit antibody (Amersham, Little Chalfont, UK). The DNA stain was either performed using TO-PRO-3 (Molecular probes, Eugene, Oreg.). The sections were analysed on a Zeiss axioplan 2 confocal microscope.

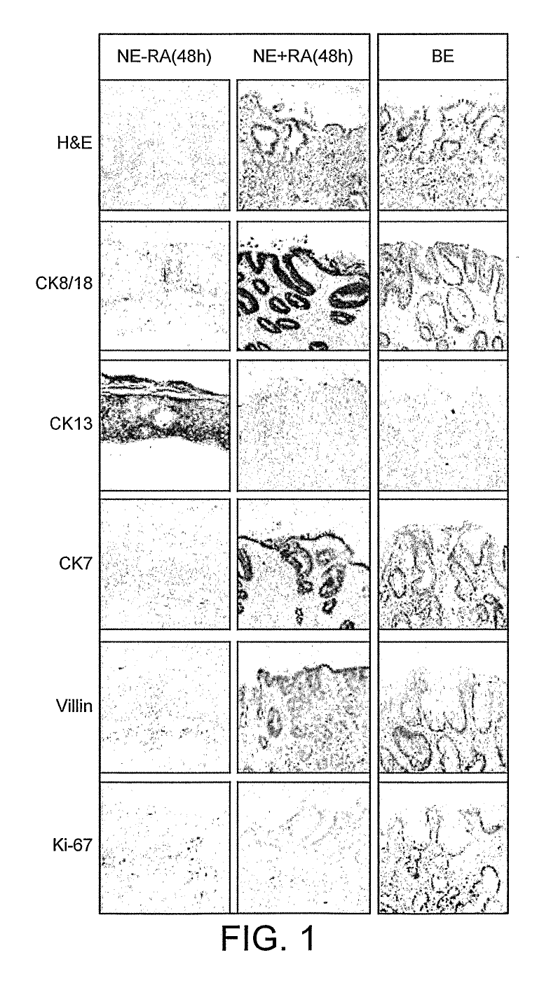

Morphological and immunohistochemical analysis demonstrates that after 48 hours, 8/31 (26%) of the squamous tissue treated with retinoic acid has a glandular phenotype with a villiform surface and mucosal glands (FIG. 1). Multivariate analysis does not reveal any difference in the age, sex or endoscopic diagnoses of the patients from which successful compared with unsuccessful retinoic treated samples are obtained. The retinoic acid induced glandular tissue expresses cytokeratins CK8/18 and CK7 but not CK13 resembling the native Barrett's epithelium. In addition, villin, a brush border protein previously used as a differentiation marker in Barrett's oesophagus, is strongly induced in the treated tissue.

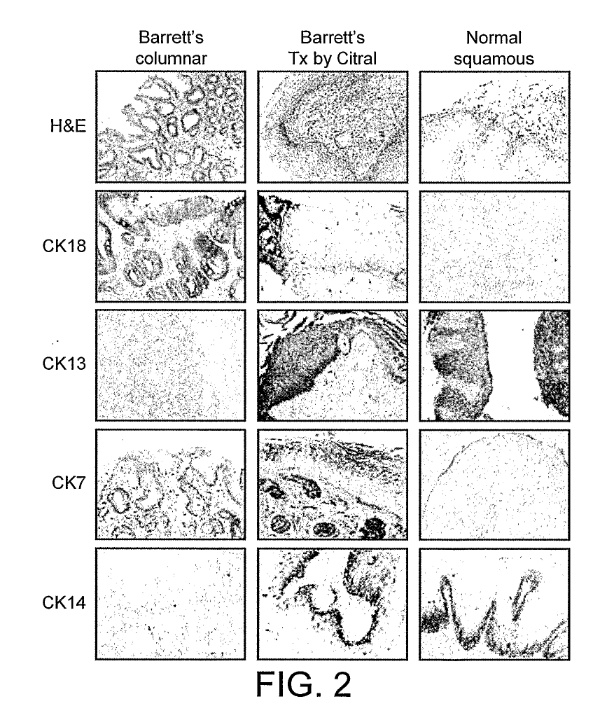

In light of these findings, to demonstrate that inhibition of retinoic acid activity could reverse the columnar phenotype, Barrett's oesophagus biopsies were treated according to the present invention by culture with the retinoic acid inhibitor citral. After 48 h 5/10 (50%) Barrett's explants had evidence of a squamous phenotype (FIG. 2). The villiform surface could be observed to be sloughed off with remnant glandular structures underlying the squamous mucosa. The immunohistochemical characteristics resembled native squamous oesophagus with expression of CK13 and CK14, but not CK8/18. CK 7 which is usually expressed in the glandular mucosa, was slectively expressed in the superficial layers of the neo-squamous epithelium (FIG. 2). For both the retinoic acid and the citral treated tissues Multivariate analysis did not reveal any difference in the age, sex or endoscopic diagnoses of the patients from which successful compared with unsuccessful retinoic acid or citral treated samples were obtained.

The rapid time course of this phenotypic change suggests a process of transdifferentiation rather than proliferation of a pre-existing glandular compartment.

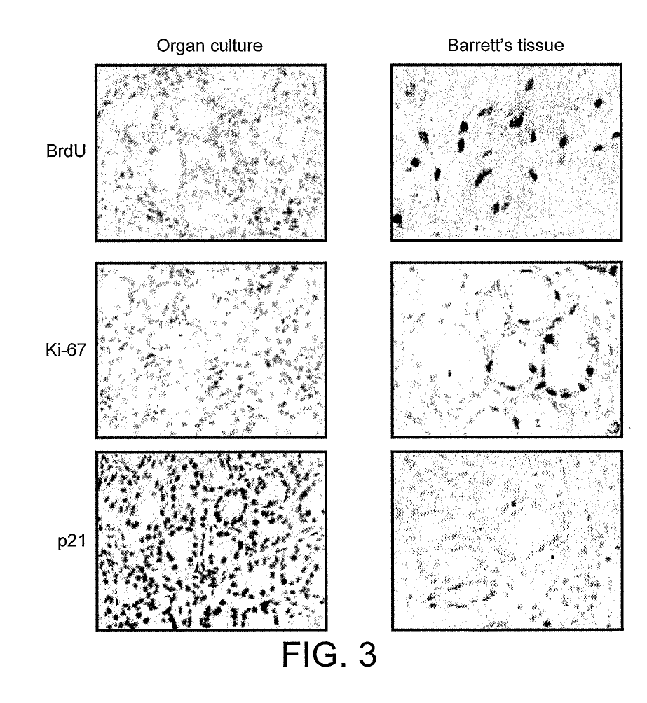

This was confirmed by the lack of BrdU incorporation and reduced Ki-67 staining in both the retinoic acid and the citral transformed tissues compared with their native counterparts. Moreover, massive over-expression of p21 also suggests cell cycle arrest (FIG. 3).

### EXAMPLE 2

### Origin of Glandular Mucosa

In order to determine whether the glandular mucosa originated from either the epithelial or the mesenchymal compartments, the endoscopic squamous biopsy is separated into epithelium and mesenchyme prior to culture. RT-PCR of these cultures is performed as follows.

RT-PCR. A cryosection was performed from the RA cultured biopsy prior to RNA extraction to confirm that glandular differentiation had occurred. Total RNA was isolated using Trizol Reagent (Invitrogen, Paisley, UK). 2 ug of RNA was reversed transcribed, and 2 μl of cDNA amplified. Amplification conditions consisted of 30 cycles of 94° C. for 30 s, 55° C. for 30 s and 72° C. for 45 s for CK7, CK 13 and CK8/18 as previously described (Xu et al. Biochem Biophys Res Commun 287, 47-55 (2001).

The amplification of GAPDH was 94° C. for 45 s, 60° C. for 45 s and 72° C. for 1 min. PCR products were analysed on 1.5% agarose gels, stained with ethidium bromide and quantified by densitometry.

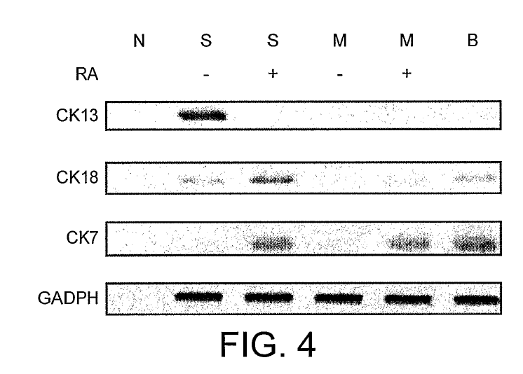

The results as illustrated in FIG. 4 confirm that the squamous epithelial compartment had been successfully removed prior to culture. Furthermore, the morphological markers normally expressed in Barrett's oesophagus are shown to be positive by RT-PCR following retinoic acid induced differentiation of the mesenchymal.

### EXAMPLE 3

### Temporal Characteristics

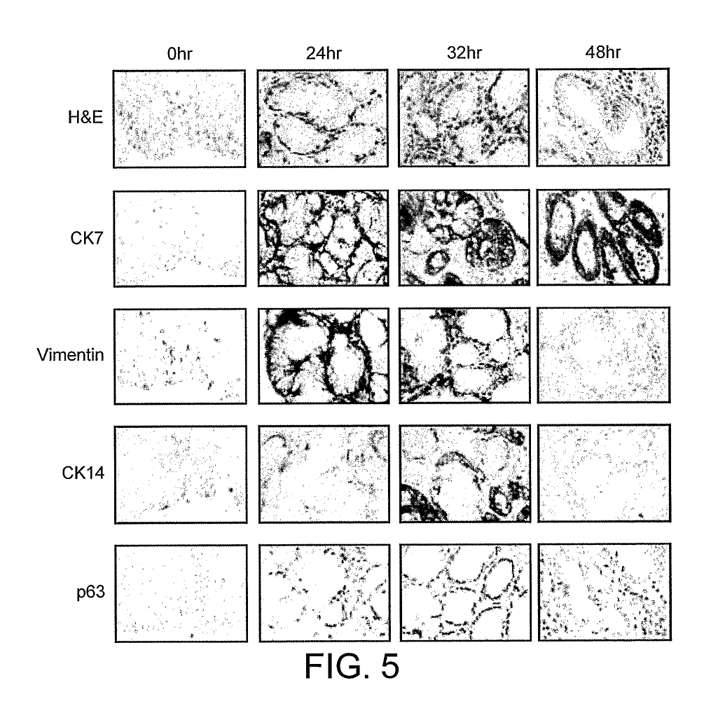

The temporal evolution of retinoic acid-induced glandular differentiation is examined by culturing the biopsies for different time periods (FIG. 5, 6a). Histological examination of the consecutive changes reveals that after 24 hours the glands have an immature morphological appearance with positive immunohistochemical staining for vimentin and negative CK8/18. Over the next 24 hours the vimentin positivity gives way to CK8/18 staining and, throughout the culture period there are cells within the glands which stain positively for putative stem cell markers p63 and CK14. Moreover, it can be seen by dual-labelling Immunoflurescence that at the early time point (24 hours) there is co-localisation of vimentin and CK8/18 which then separates out at 32 and 48 hours into CK 8/18 positive glands and vimentin positive stroma.

Thus, to summarise, after 48 h of retinoic acid treatment in culture, 8/31 (26%) squamous explants had a glandular phenotype with a villiform surface and mucosal glands. Conversely, after 48 h of citral treatment 5/10 (50%) Barrett's explants had a squamous phenotype. The immunohistochemical characteristics closely resembled the native tissue. Lack of BrdU incorporation and reduced Ki67 staining suggest that this phenotypic change is proliferation independent. The Barrett's metaplastic transformation could be induced from mesenchyme alone and dual-labelling immunofluoresence demonstrated co-localisation of vimentin and CK8/18 at 24 h which then separated into CK 8/18 positive glands and vimentin positive stroma.

Thus, we have demonstrated that citral reverses columnar lined Barrett's epithelium according to the present invention.

### EXAMPLE 4

### Cell Lineage Characteristics

The ulcer-associated cell lineage (UACL) is the prototype repair lineage which occurs in the gastrointestinal tract in response to chronic damage. It is classically manifest as groups of acinar structures within the laminar propria of the gut associated with a duct draining onto the villus surface with a unique secretory profile containing periodic-acid Schiff positive mucin and a specific trefoil peptide profile.

The UACL is unusual in that the initial elaboration of the structure occurs without indigenous mitotic activity and appears to develop its own stem cell region. The mucin and trefoil peptide staining characteristics of the glandular epithelium in the ex vivo model according to the present invention appear to have much in common with the UACL with positive staining for TFF2 and 3 (TFF1 expression, FIG. 1).

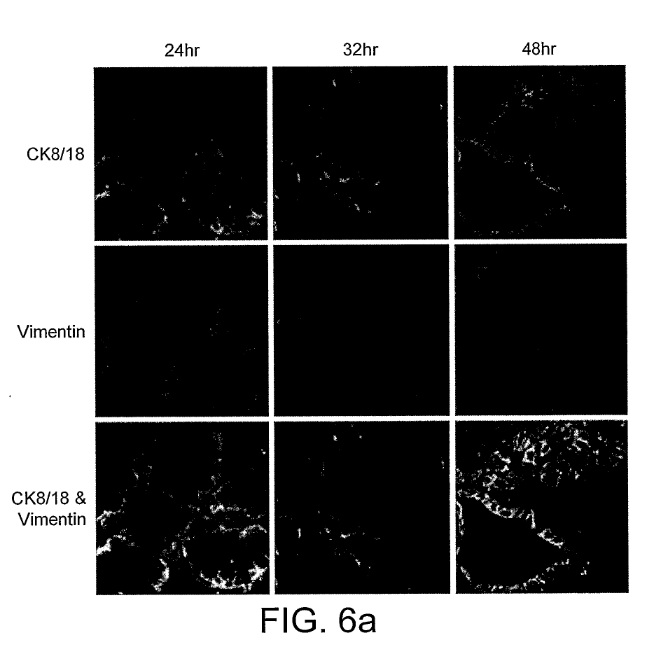

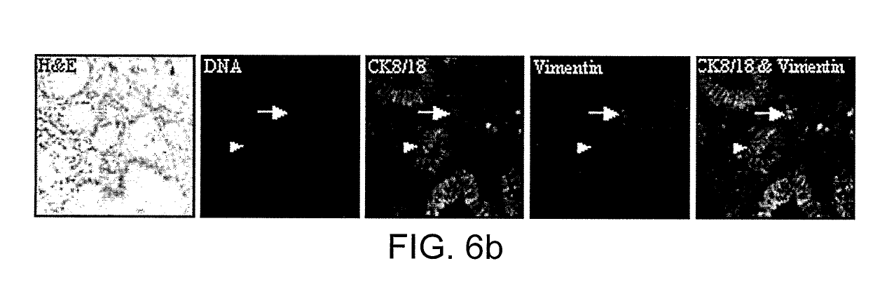

Sections of native Barrett's mucosa were also stained with CK8/18 and vimentin to determine whether there was co-localisation of these markers within any of the immature glandular structures. Whereas most of the mature, surface glands did not show co-localisation (FIG. 6b), in some deeper more immature glands co-localisation was demonstrated, in keeping with the ex vivo findings.

Previously, continuous acid exposure ex vivo was shown to increase the degree of differentiation of native Barrett's epithelium. The present invention advantageously provides induction of a glandular phenotype from adult oesophageal squamous mucosa ex vivo. The data presented here suggest that retinoic acid induces the Barrett's phenotype de novo via a mesenchymal epithelial transition pathway.

The ex vivo model system of the present invention precludes the possibility that the glandular differentiation has originated from pluripotent stem cells at the gastro-oesophageal junction (transitional zone) since the squamous biopsies were taken at least 2 cm away firm this region. It would also seem unlikely that there has been transdifferentiation from the squamous epithelial layer along an abnormal cell lineage pathway since culture of the mesenchymal compartment alone is sufficient for glandular differentiation to occur.

Without wishing to be bound by theory, the question therefore arises as to which cells within the mesenchyme give rise to this columnar epithelium. It is possible, that as previously suggested, the glandular tissue may arise from the stem cells located in the region of oesophageal glands and associated ducts. Support for this theory has come from observational studies of neo-columnar lined epithelium generated within animal models of Barrett's oesophagus. In addition, neo-squamous epithelium has been observed extending from the submucosal gland duct following photodynamic therapy. The system of the present invention does not exclude this possibility.

However, the phenotypic change cannot have arisen from proliferation of a small number of stem cells or from a native submucosal duct because a maximum of two cycles of cell division would be possible during the incubation period and, in contrast, the data suggests that there is cell cycle arrest (FIG. 3). Furthermore, the dual labelling studies suggest a transition from mesenchyme to epithelium at 24 hours of culture (FIG. 6a). Hence, the data favour a mesenchymal-epithelial transition, consistent with accepted mechanisms for retinoic acid-induced glandular differentiation and normal embryonic development.

In addition, it appears that in the model system of the invention the glandular structures appear to share some of the mucin characteristics of the UACL. Without wishing to be bound by theory, we suggest that the initial origin of the glandular epithelium is mesenchymal with further differentiation along an UACL.

The retinoic-acid induced columnar type epithelium of the model according to the present invention does not show any features of intestinalisation. There is a villiform surface with a brush border as evidenced by positive villin staining, but at 48 hours there is no evidence of goblet cells or other mucins from Alcian blue-PAS staining. The model may advantageously be used to determine whether further differentiation could be induced by exposure to specific components of refluxate in an ex vivo or xenograft system according to the theory of sequential specialisation of Barrett's oesophagus.

The existence of immature glands with co-localisation of CK8/18 and vimentin in native Barrett's mucosa biopsies (FIG. 6b), similar to the appearances of retinoic acid treated squamous epithelium at 24 hours (FIG. 6a), demonstrate that the in vitro system of the present invention is a robust model.

Interestingly, in this in vitro model an excess of vitamin A, induces the metaplastic (and potentially premalignant) change. Although altered expression of nuclear retinoid receptors is associated with malignant transformation of human cells, the role of altered retinoic acid pathways in the oesophagus has not been well characterised. There may be altered levels of retinoic acid receptors (RAR) in Barrett's oesophagus compared to adjacent normal mucosa and further alterations in the Barrett's-dysplasia-carcinoma sequence. This may be advantageously investigated using the model of the present invention in order to further understand the in vivo significance of retinoic acid in Barrett's oesophagus as opposed to the downstream signalling pathways which may be activated in vivo independently of retinoic acid stimulation. For example, the cdx2 intestinal homeobox gene which may be induced by retinoic acid has also been shown to be activated by chronic acid exposure in a long-term culture of mouse oesophageal keratinocytes, even though this did not lead to any phenotypic alteration. The model of the present invention now renders more detailed investigation of these issues possible.

Thus, by comparison of the ex vivo model of the present invention to Barrett's oesophagus in vivo, the value of the model of the present invention is demonstrated.

### EXAMPLE 5

### Determining Effects of Specific Retinoic Acid Antagonists on Oesophageal Cell Phenotype

Organotypic Culture and Organ Culture

Prepare 500 ml medium: MCDB 153 medium supplemented with FCS, 20 ng/ml EGF, 140 μg/ml BPE, 5 μg/ml insulin, 5 μg/ml transferrin and 5 ng/ml selenium. Grow 1×10⁵ cells derived from human Barrett's oesophageal epithelium transduced with hTERT (CP-52731) in supplemented media in a T75 flask.

When confluency has been reached pre-treat cells with pharmacological agents according to protocol below—each test culture must be carried out in triplicate and repeated on at least 2 separate occasions. Paired control cultures with plain media with no additives.

Prepare raft culture from a collagen matrix containing 100% rat tail collagen, fetal bovine serum, 1× DMEM medium, reconstituted buffer and immortalized human fibroblast (hTERT-BJ1, 1×10⁶) in Nunc 6-well dish.

Seed 1×10⁶ cells onto a raft culture and incubated at 37° C. with a air-liquid phase to establish the multilayer epithelial component. Medium was changed every three days.

Organ Culture

Prepare organ culture medium: Medium 199 supplemented with 10% FCS, 1 μg/ml of insulin.

For pharmacological manipulations add to the organ culture media from the start of the experiment. each test culture must be carried out in triplicate and repeated on at least 2 separate occasions. Paired control cultures with plain media with no additives. Arrange collection of endoscopic biopsy samples.

Place biopsies on a sterilized stainless steel grid within a 6 ml Petri dish and culture in 3 ml organ culture media per petri dish. The culture medium should just cover the surface of the biopsy. Place the petri dishes on racks within organ culture tank and bubble with oxygen to a final concentration of 95%. Culture with gas/liquid interface for a maximum of 72 hours. If any manipulations are carried out within this time period the tank must be re-gassed.

Pharmacological Manipulation

Pretreated confluent cells with or without 2 mM Citral for 30 min and then seed in raft culture with above medium. Citral (20 mM) was added from the 5^{th} day of culture. In organ culture, 20 mM Citral was supplemented in the medium during entire incubation period.

The organotypic culture is incubated for 10 days. On the end of culture, the medium was taken out and replaced with 10% formalin in the well for fixation directly; Following organ culture at 24, 32 and 48 hours remove biopies from grid and fix immediately in 10% formalin or snap-frozen in liquid nitrogen.

Histochemical Stains and Immunohistochemistry

Processed fixed tissues for paraffin embedding. Cut 5-μm sections. Stain with hematoxylin and eosin.

For characterizing the cultured tissues standard immunohistochemistry protocols were used with the following primary mouse monoclonal antibodies raised against the human epitopes:

CK7 (1:50), CK13 (1:200), CK8/18 (1:40) CK14 (1:40), vimentin (1:50), p63 (1:25) (Novocastra, Newcastle Upon Tyne, UK), villin (1:750) (Chemicon, Harrow, UK) and Ki-67 (1:100) (DakoCytomation, Ely, UK). Anti-BrdU (1:40) (AbCam, Cambridge, UK).

The avidin-biotin and peroxidase method and a hematoxylin counterstain were used. Negative controls were performed for each slide by omission of the primary antibody.

### EXAMPLE 6

### Temporal Events in Retinoic Acid Signalling

It is demonstrated herein that a columnar phenotype resembling Barrett's oesophagus can be induced by supraphysiological concentrations of All-trans retinoic acid treatment according to the present invention. In particular this effect is demonstrated in vitro in the above examples. Furthermore, a squamous phenotype can be induced by the retinoic acid antagonist citral. (Citral is a retinoic acid synthesizing antagonist.)

This demonstration highlights the in vivo significance of retinoic acid in the sequential disease process of Barrett's oesophagus, dysplasia and cancer as disclosed herein.

Methodology for Cell Lines

Cell lines are seeded in 6-well plate (80% confluence) and cultured for 24 hours by adding retinol (100 nM) in individual medium (devoid of serum). Then medium (100 ul) is taken to 96-well plate seeded with 3T3 RARE-SEAP reporter cells and incubated for 24 hours. SEAP is determined. Relative RA content in medium (biosynthesis) is calculated by comparison with standard curve using serial RA concentration and then adjusted by total protein content in cells (ug). Non-dysplastic Barrett's cells (BAR) show highest RA biosynthesis. Het1a & NES: immortalized squamous oesophageal epithelia; Go, Gi, Ch: high-grade dysplastic Barrett's cell lines.

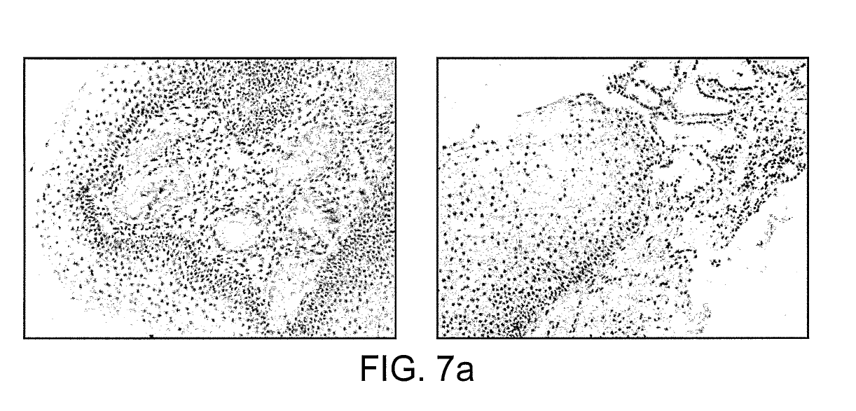

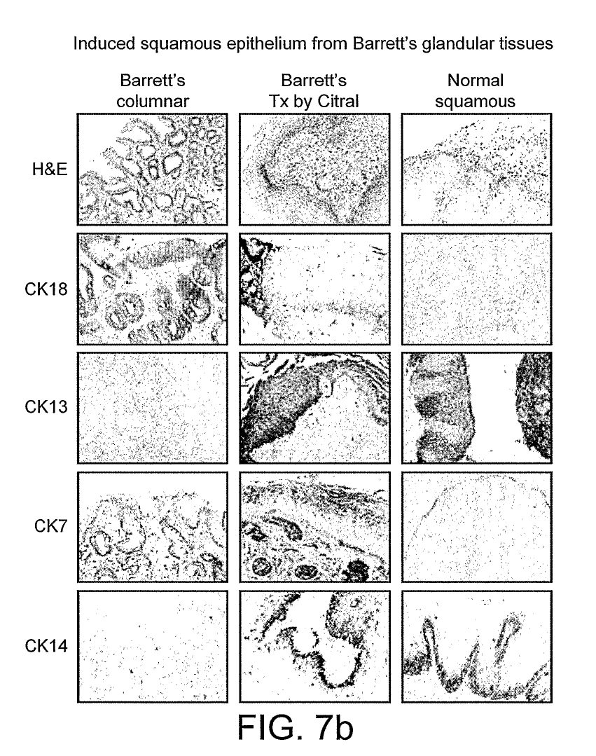

Thus, in this example it is shown that retinoic acid biosynthesis is maximal in early Barrett's (non-dysplastic) when compared with normal squamous or dysplastic epithelia in a cell line based system (FIG. 8). Moreover, this effect is also demonstrated in human tissues using biopsies (FIG. 9).

Methodology for Biopsies

Biopsied tissues are processed in organ culture (95% oxygen with air-liquid interface), retinol is added (10⁻⁶M) in medium and cultured for 18 hrs. 100 ul of the medium is taken to 3T3 pRARE-SEAP reporter cell (seeding in 96-well plate) and incubated for 24 hours. Secreted alkaline phosphatase is determined by Great EscAPe Assay System. Relative retinoic acid content in medium (biosynthesis) is calculated by comparison with standard curve using serial RA concentration and then adjusted by total protein content in tissues (ug). RA biosynthesis is highest in Barrett's (BE) (p<0.001), while there is no difference between normal epithelium (NS) and cancerous epithelium (AdenoCa)

Thus it is demonstrated that in oesophageal biopsies the RA biosynthesis is maximal in Barrett's epithelium (FIG. 9), consistent with the therapeutic interventions taught herein.

### EXAMPLE 7

### Role of Cdx2

One candidate mechanism for the phenotypic alteration occurring with retinoic acid treatment is via the homeobox gene Cdx2 which is involved in the determination of cell fate. Up-regulation of Cdx2 has been implicated in the pathogenesis of intestinal metaplasia. In addition, exposure to exogenous acid can induce Cdx2 expression in squamous epithelium. This example addresses Cdx2 involvement in the processes and therapies of the present invention.

Methodology

A Cdx2 expression plasmid is constructed by inserting full-length Cdx2 CDS into pCDNA3.1 His C and verified by sequencing. BAR and Het1a cells are transfected transiently by pCDNA Myc-His/Cdx2 using Fugene 6. Retinol (100 nM) is added to medium 48 hours later and cultured for 24 hrs. The medium is taken to RA reporter cells and incubated for another 24 hours. SEAP is checked. Western blotting shows expression of recombinant protein by His-tag antibody in transfected cells. Cdx2 overexpression enhances biosynthesis of RA in normal squamous (Het1a) and non-dysplastic Barrett cells (BAR).

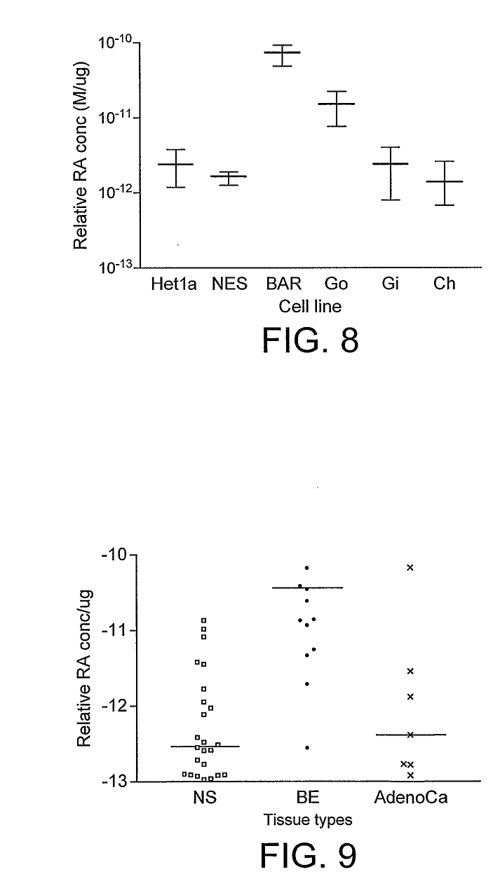

FIG. 10 shows that over-expression of Cdx2 in vitro results in increased retinoic acid signalling. BAR is Barrett's cell line and HET1 is a normal squamous oeosphageal cell line. Transfected cells in graph are denoted by “/CDX2”.

Thus we show that over-expression of Cdx2 in vitro results in increased retinoic acid signalling. Hence, wihout wishing to be bound by theory, it is consistent with the teaching of the present invention that in vivo Cdx2 may endogenously trigger retinoic acid signalling. Thus in one aspect the present invention provides a method for treating or preventing Barrett's oesophagus comprising inhibiting and/or down-regulating Cdx2, and provides the use of a Cdx2 inhibitor in the manufacture of a medicament for the prevention or treatment of Barrett's oesophagus. The inhibitor may be any suitable inhibitor such as an RNAi designed to inhibit Cdx2 expression.

### EXAMPLE 8

### Downregulation of CYP26A1

On checking the responsible enzymes for the retinoic acid biosynthesis, we found that CYP26A1 is down-regulated or even absent in early Barrett's epithelium. This is the most important degrading enzyme for metabolism of the protein-bound retinol in the excess of retinal. Hence, without wishing to be bound by theory, it might be the reason for the increased retinoic acid synthesis in early Barrett's tissues. In any case, whether or not a RA antagonist acts via CYP26A1, treatment of Barrett's oesophagus by downregulation of CYP26A1 forms a part of the present invention as explained herein.

Methodology

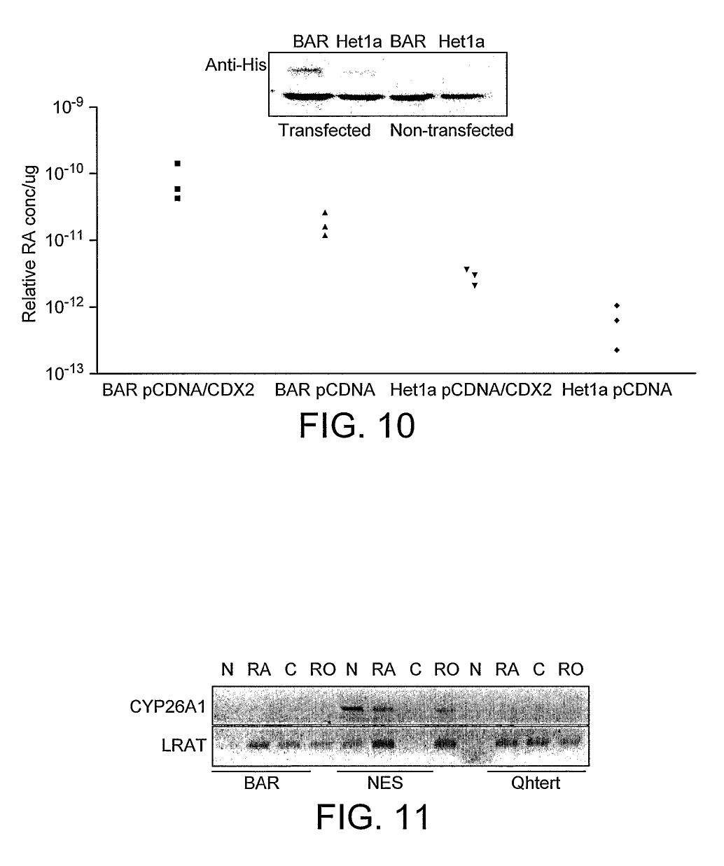

Expression of CYP26A1 in Barrett's (BAR, Qhtert) and squamous cell line (NES) is examined by RT-PCR. The results are shown in FIG. 11. CYP26A1 is normally expressed in squamous cells but down-regulated or even absent in Barrett's cells. Its regulation is not induced by adding retinoic acid (RA) or retinol (RO), but it is down-regulated in squamous cells by adding citral (C). The control when nil is added is denoted by N. The LRAT is the enzyme for retinyl ester formation from excessive retinol.

Thus these data support the use of a retinoic acid antagonist in the manufacture of a medicament for the downregulation of CYP26A1, since it is clearly shown that an antagonist such as citral has this effect, and the use of a retinoic acid antagonist in the downregulation of CYP26A1 is demonstrated. It will be appreciated that the corresponding use of an inhibitor of CYP26A1 activity in the manufacture of a medicament for the treatment or prevention of Barrett's oesophagus (Barrett's mucosa), and a method of treating Barrett's oesophagus in a subject comprising administering to said subject an effective amount of a CYP26A1 inhibitor are thus encompassed by the present invention.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. (canceled)

2. A method for the treatment or prevention of ectopic development of columnar epithelium comprising administering to a subject in need thereof a composition comprising retinoic acid antagonist.

3. (canceled)

4. (canceled)

5. A method of reducing the proliferation of ectopic columnar epithelium in a subject comprising administering to said subject in need thereof a composition comprising retinoic acid.

6. (canceled)

7. A method according to claim 2, or claim 5, wherein the retinoic acid antagonist is an aldehyde dehydrogenase inhibitor.

8. A according to claim 7 wherein the aldehyde dehydrogenase inhibitor is a competitive inhibitor of aldehyde dehydrogenase.

9. A method according claim 2, or claim 5, wherein the retinoic acid antagonist is citral.

10. A method according to claim 2, or claim 5, wherein the retinoic acid antagonist is a retinoic acid receptor antagonist.

11. A method of treating Barrett's oesophagus in a subject comprising administering to said subject an effective amount of retinoic acid antagonist.

12. A method of treating Barrett's oesophagus in a subject comprising administering to said subject an effective amount of a CYP26A1 inhibitor.

13. A method according to claim 11 or claim 12 wherein said antagonist comprises citral.

14. (canceled)

15. A method for the induction or maintenance of columnar epithelium comprising adding retinoic acid to columnar epithelial cells in an amount effective to promote said induction or maintenance.

16. A method of promoting conversion of squamous epithelium to columnar epithelium comprising contacting said squamous epithelium with retinoic acid.

17. (canceled)
